(19) United States
(12) Patent Application Publication
(10) Pub. No.: US 2006/0204530 A1
(43) Pub. Date: Sep. 14, 2006

(54) Benzoyl peroxide compositions and methods of use

(75) Inventors: Jose E. Ramirez, Trumbull, CT (US); Joseph R. Faryniarz, Middlebury, CT (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: JR CHEM, LLC

(21) Appl. No.: 11/373,538

(22) Filed: Mar. 10, 2006

Related U.S. Application Data
(60) Provisional application No. 60/660,386, filed on Mar. 10, 2005.
(60) Provisional application No. 60/695,223, filed on Jun. 29, 2005.

(51) Int. Cl. A61K 8/35; A61K 8/36; A61K 31/192

(52) U.S. Cl. 424/401; 514/568

(57) ABSTRACT

Solutions of benzoyl peroxide, in one or more solvents are provided that are suitable for direct topical application to the skin of a user or can be formulated into a product that is suitable for topical application to the skin of a user. In embodiments, the solutions include benzoyl peroxide and optionally additional active or inactive ingredients, and a solvent or mixture of solvents in which the benzoyl peroxide is prepared as a clear solution at 25° C. Methods of using the solutions are also described. Product formulations such as emulsion are also described. The solutions and product formulations can increase the efficacy and percutaneous absorption of benzoyl peroxide.

### RELATED APPLICATION

This Application claims priority benefit of U.S. Provisional Application No. 60/660,386 filed Mar. 10, 2005, and U.S. Provisional Application No. 60/695,223 filed Jun. 29, 2005, both of which are herein incorporated by reference in their entirety.

### BACKGROUND

1. Technical Field

This disclosure relates to the preparation of compositions containing benzoyl peroxide in solution for topical application to human skin. These compositions are useful for the treatment and prevention of acne with greater efficacy than conventional compositions. The compositions allow benzoyl peroxide to be utilized in new product forms, and have unique processing capabilities making them useful in fields other than acne treatment. Moreover, the compositions increase the percutaneous absorption of benzoyl peroxide in skin.

2. Background of Related Art

Benzoyl peroxide is a crystalline solid with a melting point of 103° C. to 106° C. and a molecular weight of 242.22. Pure benzoyl peroxide is extremely flammable and shock sensitive and is therefore mostly used in a water wetted form, usually containing 25% water and 75% benzoyl peroxide crystals. This material is gritty and extremely hard and requires difficult processing and milling in order to be incorporated into product forms such as a dispersion. The use of a dispersion limits the formulation of clear products and sprays. Benzoyl peroxide has some solubility in certain industrial solvents, such as the aromatic solvents of benzene and toluene, which are toxic and not suitable for human use. Ketone based solvents such as methyl ethyl ketone and acetone are also known, but the flammability of these solvents as well as the ability of these solvents to irritate and strip the skin from its protective mantle make their use limited for use in pharmaceutical or cosmetic compositions. U.S. Pat. No. 5,632,996 discloses that long chain alkyl benzoates can be used to prepare fine dispersions of crystalline benzoyl peroxide without the previously required milling process. Secondly, it teaches that this combination removes the water from the crystals providing essentially an anhydrous dispersion of the benzoyl peroxide in the long chain alkyl benzoate ester. It does not teach that there is any solubility functionality for these esters and only describes products with benzoyl peroxide as dispersions.

The current uses dispersions of benzoyl peroxide particles for the treatment of Acne Vulgaris. Acne Vulgaris is believed to be caused by a number of factors, which may result in the formation of closed white heads and blackheads having open comedones. These are solid horny materials that plug the follicles and are thought to be caused by excessive follicular sebum production, usually brought on by hormonal changes. The composition of these horny masses is a tightly packed keratinized group of cells mixed in with sebum, bacteria and other skin materials. When the comedone enlarges through the accumulation of the keratinized cells, sebum, bacteria etc., pressure builds up within the follicular walls until the follicles rupture spilling these contaminates into the surrounding skin. The skin reacts with an inflammatory response which takes the form of Pustules (Pimples) for a small rupture or cystic-nodules with complete rupture.

The current practice of treating Acne Vulgaris is to treat the skin with a benzoyl peroxide dispersion of crystals. The benzoyl peroxide is used to control the P. Acne bacillus, the microbe identified as the primary infectant of the pustules. However, the use of essentially undissolved benzoyl peroxide crystals, which are larger than the microbe being treated is highly ineffective, and requires high percentages of active to gain any effectiveness. Furthermore, the crystals have extreme difficulty in penetrating into the comedone because the plug is a physical barrier and the size of the follicular opening is limited.

Accordingly, what are needed are compositions for the treatment of acne infections which are more effective than conventional compositions; compositions where the benzoyl peroxide is in solution and/or in lower concentrations; and methods of improving the processing of benzoyl peroxide such as by dissolving the material in suitable solvents. It would also be advantageous to provide processing advantages for areas outside use in the pharmaceutical and cosmetic fields.

### SUMMARY

Benzoyl peroxide compositions including solutions of benzoyl peroxide in one or more solvents are described herein. In particularly useful embodiments, the present solutions include benzoyl peroxide and optionally additional active or inactive ingredients, and a solvent or mixture of solvents in which benzoyl peroxide is prepared as a clear solution. The solutions are suitable for direct topical application to the skin of the user and/or for delivery of the benzoyl peroxide into the comedone. In embodiments, the antimicrobial is in solution such that when applied to skin the dissolved antimicrobial is in intimate contact with the cell wall of the microbe. As antimicrobial efficacy is greatest when the microbe is attacked by the antimicrobial on a molecular level, dissolved benzoyl peroxide serums and formulations containing them have been found to be effective in treating Acne Vulgaris.

In embodiments, suitable corrective compositions in accordance with the present disclosure provide a solvent vehicle formulation for the treatment of acne in which the major active ingredient is benzoyl peroxide. The benzoyl peroxide is provided in clear product forms such as serums, toners, pump or aerosol sprays, clear gels, sticks, creams, lotions and mousses. The clear product forms can be incorporated into other pharmaceutical or cosmetic product forms such as emulsions.

In addition, dermatological treatment regimens in accordance with the present disclosure can improve characteristics of a user's skin. The regimens include the application of one or more corrective benzoyl peroxide compositions in accordance with the present disclosure suitable for treating one or more skin conditions. Suitable corrective compositions include, for example, benzoyl peroxide serums and formulations for treating Acne Vulgaris.

In a related aspect, the present disclosure is directed towards treatment of skin lesions by the application of one or more corrective compositions to skin such as one or more benzoyl peroxide serums.

In another related aspect the present disclosure is directed towards increasing the percutaneous absorption of topically applied benzoyl peroxide.

These and other aspects of this disclosure will be evident upon reference to the following detailed description.

### BRIEF DESCRIPTION OF THE DRAWINGS

FIG. 1 illustrates a comparison of follicular bactericidal activity on forehead over 8 hours after a single application of composition in accordance with the present disclosure (OMP) with a 5% benzoyl peroxide gel.

FIG. 2 illustrates a comparison of follicular bactericidal activity on forehead over 8 hours after a single application of composition in accordance with the present disclosure (OMP) with a 5% benzoyl peroxide/1% clindamycin gel.

FIG. 3 illustrates a comparison of non-inflammatory lesion counts over treatment course between a composition in accordance with the present disclosure (5% benzoyl peroxide solution) (ON) with a 5% benzoyl peroxide gel/1% clindamycin sulfate composition (BN).

FIG. 4 illustrates a comparison of inflammatory lesion counts over treatment course between a composition in accordance with the present disclosure (5% benzoyl peroxide solution) (OI) with a 5% benzoyl peroxide gel/1% clindamycin sulfate composition (BI).

### DETAILED DESCRIPTION OF PREFERRED EMBODIMENTS

The present disclosure provides compositions and methods for treating acne. The system includes applying a predetermined amount of the active drug benzoyl peroxide in solution form to an area of skin in need thereof. The solvated benzoyl peroxide molecules are available to penetrate into follicles, follicle oil glands, the stratum corneum and epidermis of skin in need of treatment, including infected areas. Benzoyl peroxide saturates the targeted infected areas to destroy the P. Acne bacteria. Since the penetrated benzoyl peroxide is in solution, it is available to skin in need thereof in the most bio-effective form.

Accordingly, the present compositions provide a solvent vehicle formulation for the treatment of acne in which the major active ingredient is benzoyl peroxide. In embodiments, the active ingredients are provided in a previously unobtainable clear product forms. Such clear product forms include serums, toners, pump or aerosol sprays, clear gels, sticks, creams, lotions and mousses. The clear product forms improve the effectiveness of the ingredients applied, allowing the use of lower levels and providing quicker patient response. Also the use of these vehicle solvents in emulsion product forms allows levels of benzoyl peroxide in oil/water emulsions which exceed the solubility parameters of benzoyl peroxide in o/w systems without the solvent vehicle, resulting in a slurry type application with increased antimicrobial activity due to the solubilized fraction of the benzoyl peroxide. Also, the use of solvents that have relatively low water solubility and relatively high solubility for benzoyl peroxide will be more effective than the long chain alkyl benzoates in removing water from the crystals during processing, offering a safe handling method for other industrial uses.

In embodiments, compositions in accordance with the present disclosure include benzoyl peroxide in solution in one or more solvents. Benzoyl peroxide is normally commercially available as either pure (98% active) crystals or in a wet crystalline state containing 70 to 80% active benzoyl peroxide in 20-30% water. Any commercially available forms of benzoyl peroxide can be mixed with the disclosed solvents to form compositions in accordance with this disclosure.

The amount of benzoyl peroxide mixed with the solvent will vary depending on a number of factors, including, for example, the activity of benzoyl peroxide, the ultimate form of the product and the particular disclosed solvent employed. Generally, the benzoyl peroxide will constitute from 1 to 70 weight percent of the benzoyl peroxide/solvent mixture. In embodiments, the benzoyl peroxide constitutes from about 3.00 to about 40 weight percent of the benzoyl peroxide/solvent mixture. In embodiments, the benzoyl peroxide constitutes from about 2 to about 15 weight percent of the benzoyl peroxide/solvent mixture. In embodiments, benzoyl peroxide is present in amounts effective for treating Acne Vulgaris. Due to the increased availability of the benzoyl peroxide in the present compositions (as shown in the working examples below), the minimum effective amount of benzoyl peroxide may, in embodiments, be lower than the amounts in presently available acne treatments containing benzoyl peroxide. Thus, compositions in accordance with the present disclosure may contain 5% benzoyl peroxide and have 2 to 3 times the skin penetration than conventional 10% benzoyl peroxide compositions.

Solvents useful for preparing the present solutions include any solvent capable solubilizing benzoyl peroxide. Non-limiting examples of such solvents include short chain alkyl esters, ethers, aldehydes, ketones or alcohols of benzoic acid, benzyl alcohol, salicylic acid, phenol or phathalic acid. Other suitable solvents include aryl esters, ethers, aldehydes, ketones and alcohols of benzoic acid, benzyl alcohol, salicylic acid, phenol and phthalic acid. In certain embodiments, the compositions in accordance with the present disclosure include one or more of the following classes of solvent: alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol. Additional non-limiting examples of suitable solvents include benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzaldehyde, benzyl alcohol, ethyl benzyl alcohol, salicylaldehyde, benzyl salicylate, phenyl tolyl ketone, phenyl benzoate, phenyl ether, dibenzyl ether, benzyl benzoate, benzoic acid and 2-phenyl ethyl ester.

The amount of solvent mixed with the benzoyl peroxide will vary depending on a number of factors, including, for example, the ultimate form of the product and the particular solvent employed. Generally, the solvent will constitute from 1 to 70 weight percent of the benzoyl peroxide/solvent mixture. In embodiments, the solvent constitutes from about 10 to about 50 weight percent of the total composition. In embodiments, the solvent constitutes from about 20 to about 40 weight percent of the total composition. In embodiments, solvent is present in amounts effective for dissolving benzoyl peroxide.

In addition to the solvent in which benzoyl peroxide is soluble, the compositions in accordance with the present disclosure may contain one or more secondary solvents. Suitable secondary solvents include, for example, ethanol, acetone, dimethyl isosorbide, and glycol ethers of C₁ to C₆ alcohols with no greater than 2 moles of ethylene oxide. Suitable glycol ethers include glycol ethers of phenol with no greater than 2 moles of ethylene oxide, glycol ethers of methanol with no greater than 2 moles of ethylene oxide, glycol ethers of ethanol with no greater than 2 moles of ethylene oxide and glycol ethers of propanol with no greater than 2 moles of ethylene oxide. Non-limiting examples of such co-solvents include phenoxy ethanol, ethoxy diglycol and propylene glycol methyl ether.

The amount of secondary solvent mixed with the benzoyl peroxide/solvent mixture will vary depending on a number of factors, including, for example, the ultimate form of the product and the particular solvent and/or secondary solvent employed. Generally, the secondary solvent will constitute from 1 to 40 weight percent of the total composition. In embodiments, the secondary solvent constitutes from about 5 to about 30 weight percent of the total composition. In embodiments, the solvent constitutes from about 10 to about 20 weight percent of the total composition.

In preparing some compositions in accordance with this disclosure, benzoyl peroxide is simply mixed with the disclosed solvents, which may occur room temperature. As previously mentioned, benzoyl peroxide is normally commercially available as either pure crystals or in a wet crystalline state. Any of these or other forms of benzoyl peroxide can be mixed with the disclosed solvents to form compositions in accordance with this disclosure.

In addition to benzoyl peroxide, the present compositions may also optionally include salicylic acid, antibiotics and/or any other material in amounts effective for acne treatment. Antimicrobials which may be combined with benzoyl peroxide compositions in accordance with the present disclosure include all antibiotics, antimicrobial agents and antimicrobial peptides. Non-limiting examples of suitable antibiotics include inter alia dermatologically acceptable salts of tetracylin and tetracyclin derivatives, gentamycin, kanamycin, streptomycin, neomycin, capreomycin, lineomycin, paromomycin, tobramycin, erythromycin, triclosan, octopirox, parachlorometa xylenol nystatin, tolnaftate, miconazole hydrochloride, chlorhexidine gluconate, chlorhexidin hydrochloride, methanamine hippurate, methanamine mandelate, minocycline hydrochloride, clindamycin, cleocin, b-lactam derivatives such as aminopenicillin and mixtures thereof. In embodiments, a combination of chlorhexidin gluconate and triclosan is suitable for use herein. In embodiments antimicrobial agents that may be used in accordance with the present disclosure either alone or in combination include for example benzoyl peroxide and salicylic acid.

The amount of antibiotic mixed with the benzoyl peroxide/solvent mixture will vary depending on a number of factors, including, for example, the ultimate form of the product and the particular solvent and/or secondary solvents employed. Generally, the antimicrobial will constitute from 1.0 to 30 weight percent of the total composition. In embodiments, the antibiotic constitutes from about 0.1 to about 5 weight percent of the total composition.

In embodiments, benzoyl peroxide can be added to the solvents to form a mixture at room temperature, e.g., at a temperature of 25 to about 27° C. Additionally, the benzoyl peroxide/solvent mixture also can be added to other ingredients to form desired products, e.g., emulsions, lotions, creams or gels at low temperatures. In these processes, since benzoyl peroxide is never in contact with substantial heat, the possibility of decomposition or fire is greatly reduced. However the key difference between the disclosed prior art and the present compositions is that the benzoyl peroxide will actively go into solution at levels as high as 10% by weight of the total formulation. These solutions can offer new clear products of increased efficacy.

Furthermore, the higher affinity of benzoyl peroxide to the disclosed solvents offers improved method for preparing anhydrous benzoyl peroxide without subjecting the composition to any heat during processing. For example, when benzoyl peroxide-wet crystals containing 25% water are mixed with solvents in accordance with the present disclosure, the solvents (which solubilize the benzoyl peroxide) replace water in the process of changing the crystalline benzoyl peroxide into a solution, water can readily be separated. Secondly, if levels of benzoyl peroxide are desired that exceed the solubility parameters of the solvents a saturated solution in conjunction with a fine soft benzoyl peroxide slurry is formed. This composition can then be filtered to remove the water from the composition, thereby providing a fine textured, substantially, water reduced benzoyl peroxide paste/saturated solution composition. The benzoyl peroxide solutions offer a way to use benzoyl peroxide in other industrial applications where anhydrous solutions will offer advantages over dispersions. In addition, the present process provides the advantage of more effective water removal if required for the desired application.

In embodiments thickeners and/or rheology modifiers such as fumed silica may be added to the solutions to increase the viscosity of the compositions and/or gel the compositions. In embodiments, the thickener and/or rheology modifiers constitute from about 0.1 to about 10 weight percent of the total composition.

As mentioned above, the benzoyl peroxide corrective compositions in accordance with the present disclosure can be added to product forms. Suitable product forms include solutions, emulsions (including microemulsions), suspensions, creams, lotions, gels, sticks, powders, or other typical solid or liquid compositions used for treatment of skin. Such compositions may contain antimicrobial, cooling, solvent constituents and, other ingredients typically used in such products, such as moisturizers and hydration agents, penetration agents, preservatives, emulsifiers, natural or synthetic oils, surfactants, detergents, gelling agents, emollients, antioxidants, fragrances, fillers, thickeners, waxes, odor absorbers, dyestuffs, coloring agents, powders, viscosity-controlling agents and water, and optionally including anti-itch actives, botanical extracts, conditioning agents, darkening or lightening agents, glitter, humectants, mica, minerals, polyphenols, silicones or derivatives thereof, sun blocks, vitamins, and phytomedicinals. In embodiments, product forms have antioxidants to promote stability of the formulation. Non-limiting examples of suitable antioxidant compositions for use in accordance with the present disclosure are further described in U.S. Patent Application No. 60/660,387 filed on Mar. 10, 2005, entitled Stable Organic Peroxide Compositions (herein incorporated by reference in its entirety).

In embodiments, compositions in accordance with the present disclosure are useful in the formation of oil-in-water emulsion product forms. Conventional emulsion formulation typically requires mixing the aqueous phase ingredients and the dispersant with heating until a uniform solution or dispersion is obtained (optionally in several stages), mixing the organic phase ingredients with heating until a uniform solution or dispersion is obtained (also optionally in several stages), then adding the aqueous phase to the organic phase with agitation (e.g. stirring or other shearing or heating technique) to form an oil-in-water emulsion of the two phases. However, heating steps are problematic in that heat decomposes organic peroxides such as benzoyl peroxide. In embodiments, emulsion compositions in accordance with the present disclosure are capable of a low temperature blending and shearing techniques that do not require an intensive heating step of 70° C. or above. Accordingly, such blending can occur at room temperature.

In some emulsion embodiments, the aqueous phase constituting the dispersion medium may include any suitable surfactant, humectant, suspending agent, and/or buffer systems, and combinations thereof suitable for combining with benzoyl peroxide.

Non-limiting examples of suitable surfactants include natural compounds, such as phospholipids and cholates, or nonnatural compounds such as: polysorbates, which are fatty acid esters of polyethoxylated sorbitol; polyethylene glycol esters of fatty acids from sources such as castor oil; polyethoxylated fatty acid, e.g. stearic acid; octylphenolpoly(ethyleneglycolether); polyethoxylated isooctylphenol/formaldehyde polymer; poloxamers, e.g., poly(oxyethylene)poly(oxypropylene) block copolymers; polyoxyethylene fatty alcohol ethers ; polyoxyethylene nonylphenyl ethers; polyoxyethylene isooctylphenyl ethers; and SDS.

In embodiments, non-limiting examples of suitable mixtures of surfactant molecules, including mixtures of surfactants of different chemical types, are acceptable. Surfactants should be suitable for cosmetic or pharmaceutical administration and compatible with the benzoyl peroxide to be delivered. Other non-limiting examples of surfactants include phospholipids such as phosphatidylcholines (lecithins), including soy or egg lecithin. Other suitable phospholipids include phosphatidylglycerol, phosphatidylinositol, phosphatidylserine, phosphatidic acid, cardiolipin, and phosphatidylethanolamine. The phospholipids may be isolated from natural sources or prepared by synthesis.

Non-limiting examples of suitable suspending agents that are made of the following constituents: polyacrylamide, C13-14 isoparafin & laureth 7; C13-14 isoparaffin, mineral oil, polyacrylate, polyacrylamide and ethoxylated sorbitan ester; acrylamide/sodium acryloyldimethyl taurate copolymer, isohexadecane and ethoxylated sorbitan ester; and combinations thereof. However any cosmetically or pharmaceutically acceptable suspending agent suitable for combining with organic peroxide may be used.

Non-limiting examples of suitable humectants include glycerin; however any material capable of obtaining moisture may be added provided it is stable with benzoyl peroxide.

The products formulated with the present solutions can be packaged in any type of container within the purview of those skilled in the art, including, but not limited to bottles, tubes, pump type, roll-ons, daubers, wipes, and the like.

The benzoyl peroxide compositions in accordance with the present disclosure can be topically applied to skin in need of improvement in order to reduce or eliminate undesirable skin conditions. As used herein the word “treat,” “treating” or “treatment” refers to using the compositions of the present disclosure prophylactically to prevent outbreaks of undesirable skin condition such as Acne Vulgaris, or therapeutically to ameliorate an existing undesirable skin condition. A number of different treatments are now possible, which reduce and/or eliminate skin conditions such as Acne Vulgaris.

As used herein “skin condition” refers to any detectable skin manifestations caused by one or more pathogens or microbes. Such manifestations can be compounded due to a number of factors such as, for example, chronological aging, environmental damage, and/or other diseased or dysfunctional state. Non-limiting examples of such manifestations include the development of skin lines, crevices, bumps, comedones, craters, scaliness, flakiness and/or other forms of skin unevenness, roughness, or mottled appearance. It is understood, that the listed skin conditions are non-limiting and that only a portion of the skin conditions suitable for treatment in accordance with the present disclosure are listed herein.

In embodiments, compositions for use in accordance with the present disclosure contain benzoyl peroxide in an effective amount to improve undesirable skin conditions. As used herein “effective amount” refers to an amount of a compound or composition having benzoyl peroxide constituents in accordance with the present disclosure that is sufficient to induce a particular positive benefit to skin having a skin condition. The positive benefit can be health-related, or it may be more cosmetic in nature, or it may be a combination of the two. In embodiments, the positive benefit is achieved by contacting skin with a combination of solvated benzoyl peroxide, and/or one or more antibiotic constituents, to improve a skin condition such as Acne Vulgaris.

The particular benzoyl peroxide concentration in the compositions generally depends on the purpose for which the composition is to be applied. For example, the dosage and frequency of application can vary depending upon the type and severity of the skin condition.

Treatments in accordance with the present disclosure contact skin with benzoyl peroxide in an effective amount to improve acne related skin conditions. In embodiments, patients are treated by topically applying to skin suffering from an acne related condition, one or more benzoyl peroxide compositions. The active ingredient is applied until the treatment goals are obtained. However, the duration of the treatment can vary depending on the severity of the condition. For example, treatments can last several weeks to months depending on whether the goal of treatment is to reduce or eliminate an acne related skin condition.

Treatments in accordance with the present disclosure increase the percutaneous absorption of benzoyl peroxide by contacting skin with an effective amount of one or more benzoyl peroxide composition in accordance with the present disclosure. In embodiments, subjects are treated with dissolved benzoyl peroxide by topically applying the dissolved mixture to skin. The benzoyl peroxide may be applied until the absorption goals are obtained. In embodiments, the percutaneous absorption of the benzoyl peroxide is increased compared to application of the undissolved benzoyl peroxide compositions. Accordingly, higher concentrations of benzoyl peroxide can be found in the epidermis, stratum corneum, and stratum corneum surface in a single application, than when compared to formulations utilizing undissolved benzoyl peroxide.

The following non-limiting examples further illustrate compositions, methods, and treatments in accordance with the present disclosure. It should be noted that the disclosure is not limited to the specific details embodied in the examples.

### EXAMPLES 1-6

Clear serums were formulated having the following compositions:

| | EXAMPLES # | | | | | |
|---|---|---|---|---|---|---|
| | 1 | 2 | 3 | 4 | 5 | 6 |
| Dimethyl Isosorbide | 21.6% | 21.6% | 21.6% | 25.0% | 25.0% | 25.0% |
| Benzoic acid, 2-phenyl ethanol ester | 21.6% | 21.6% | 21.6% | 24.0% | 26.0% | 24.0% |
| Benzyl Peroxide wet with 26% water | 3.14% | 3.14% | 3.14% | 3.14% | 3.14% | 3.14% |
| Benzoic acid | 5.0% | 5.0% | 5.0% | 5.0% | 5.0% | 5.0% |
| Salicyclic acid | 2.0% | 2.0% | 2.0% | 2.0% | 2.0% | 2.0% |
| Benzyl Alcohol | 21.66% | 21.66% | 21.66% | 25.86% | 25.86% | 25.86% |
| Propylene glycol monomethyl ether | 25.0% | — | — | 15.0% | — | — |
| Ethanol | — | — | 25.0% | — | — | 15.0% |

These compositions were prepared by adding benzoyl peroxide to the solvents to form a mixture at low temperatures. Then the other ingredients were added with continued mixing to provide a clear cosmetic product.

### EXAMPLES 7-18

Clear serums were formulated having the following constituents shown in percent weight of the total composition:

| | Example # | | | | | | | | | | | |
|---|---|---|---|---|---|---|---|---|---|---|---|---|
| | 7 | 8 | 9 | 10 | 11 | 12 | 13 | 14 | 15 | 16 | 17 | 18 |
| Benzyl Peroxide 98% | 10 | 10 | 10 | 10 | 8 | 8 | 8 | 8 | 8 | 8 | 8 | 6.25 |
| Dimethyl Isosorbide | | | 30 | 40 | 46 | 43.5 | 45 | 45 | 45 | 45 | 45 | 40 |
| Benzyl Benzoate | 85 | | 60 | | | | | | 18.5 | | | 42.45 |
| Acetone | 5 | 5 | | | | | | | | | | |
| Benzoic acid, 2-phenyl | | 85 | | 30 | 46 | 43.5 | 23.5 | 18.5 | — | 37 | 34 | |
| ethyl ester | | | | | | | | | | | | |
| Benzyl Alcohol | | | | 20 | | | 23.5 | 18.5 | 18.5 | | 3 | |
| Ethoxy ethanol | | | | | | 5 | | 10 | 10 | 10 | 10 | |
| Vitamin E Acetate | | | | | | | | | | | | 0.5 |
| BHT | | | | | | | | | | | | 0.8 |
| Ethoxy diglycol | | | | | | | | | | | | 10 |

These compositions were prepared by adding benzoyl peroxide to the solvents to form a mixture at room temperature. Then the other ingredients were added with continued mixing to provide a clear serum.

### EXAMPLE19

Suitable compositions can be formulated having the following constituents shown in percent weight of the total composition:

| Ingredient | Amount |
|---|---|
| Benzoyl Peroxide | 4.0-6.25% |
| Benzoyl benzoate | 35-50% |
| Dimethyl isosorbide | 30-50% |
| BHT | 0.2-5% |
| Ethoxy diglycol | 5-20% |
| volatile silicone | 0-20% |
| Fumed silica | 0-10% |

### EXAMPLES 20

An emulsion was formulated having the following composition:

| Ingredient | Amount |
|---|---|
| Water | 67.6% |
| Steareth 40 | 0.8% |
| Glycerine | 4.0% |
| Benzoyl Peroxide 74% | 7.0% |
| Benzyl benzoate | 10.0% |
| Cyclomethicone | 5.0% |
| Ethylene Diamine tetraacetic acid | 0.1% |
| disodium salt (EDTA) | |
| Stearyl alcohol | 4.0% |
| Streareth 2 | 1.5% |

This composition was prepared as follows:
Phase A—Water, glycerin, EDTA, in main vessel, heated to 75° C.
Phase B—Steareth-40, cyclomethicone, steareth-2, and stearyl alcohol in auxiliary vessel heated to 75° C. Add phase B to phase A while mixing. Start cooling.
Phase C—Benzoyl peroxide and benzyl benzoate are mixed at room temperature.
Add Phase C to above mixture of phases A and B while cooling at temperature below 40° C. Cool emulsion to room temperature.

### EXAMPLE 21

### Emulsion Formulation

| Phase A Ingredients | Amount |
|---|---|
| Benzoyl Peroxide 75% wet with water | 8.68% |
| Benzyl Benzoate | 10.00% |
| BHT | 0.4% |
| Vitamin E Acetate | 0.5% |
| Dimethyl Isosorbide | 3.00% |

Add benzoyl peroxide to container with the Benzyl Benzoate, BHT and Vitamin E

Acetate and mix for 30 minutes.

Add dimethyl isosorbide and mix for additional ten minutes.

| Phase B Ingredients | Amount |
|---|---|
| DI Water | 74.22% |
| Phenoxyethanol | 0.1% |
| EDTA disodium salt | 0.1% |
| Hydroxyethylacrylate/sodium acryloyldimethyltaurate | 3.0% |
| copolymer & squalane & polysorbate 60 | |

Mix the phase B ingredients and mix to disperse.

Under high shear mixing add the phase A and mix until uniform emulsion (oil-in-water).

Other materials with desired properties may be added, provided they are stabile with organic peroxide.

### EXAMPLE 22

### Emulsion Formulation

| Phase A Ingredients | Amount |
|---|---|
| Benzoyl Peroxide 75% wet with water | 8.68% |
| Benzyl Benzoate | 10.50% |
| BHT | 0.4% |
| Dimethyl Isosorbide | 3.00% |

Add benzoyl peroxide to container with the Benzyl Benzoate, and BHT and mix for 30 minutes.

Add dimethyl isosorbide and mix for additional ten minutes.

| Phase B Ingredients | Amount |
|---|---|
| DI Water | 74.22% |
| Phenoxyethanol | 0.1% |
| EDTA disodium salt | 0.1% |
| Hydroxyethylacrylate/sodium acryloyldimethyltaurate | 3.0% |
| copolymer & squalane & polysorbate 60 | |

Mix the phase B ingredients and mix to disperse. Under high shear mixing add the Phase A and mix until uniform emulsion (oil-in-water). Other materials with desired properties may be added, provided they are stabile with organic peroxide.

### EXAMPLE 23

This example illustrates in vitro percutaneous absorption of topical benzoyl peroxide compositions in accordance with the present disclosure in skin.

### Materials and Methods

Cumulative transdermal absorption of five formulations of C¹⁴ radioactive labeled benzoyl peroxide formulations were measured over an 8 hour period in human cadaver skin using the Franz in vitro diffusion chamber. More specifically, a 5% benzoyl peroxide formulation (meeting the U.S. Pharmacopoeia labeling requirements) in accordance with the present disclosure was compared to four compositions including: a 10% benzoyl peroxide formulation; 10% benzoyl peroxide gel; 8% benzoyl peroxide formulation; and a 6% benzoyl peroxide hydrous gel formulation. The skin was supplied by a single donor, and was free from scar tissue, holes, birthmarks, infectious disease, and other irregularities. Fifteen mg of benzoyl peroxide was applied to the skin (1.7 cm² surface area) with a rubber spatula. At 8 hours the percent of benzoyl peroxide recovered in the stratum corneum, epidermis, and stratum corneum surface was determined by calculating the percent recovery of the total amount of benzoyl peroxide applied. The total micrograms of benzoyl peroxide recovered from each location were calculated by multiplying the percent recovered by the total micrograms of product applied.

### Results

After 8 hours of treatment, the total micrograms of labeled benzoyl peroxide and the percent of the applied dose recovered in the combined epidermis, stratum corneum, and stratum corneum surface were greater with the 5% benzoyl peroxide composition in accordance with the present disclosure than with any other formulation. Table 1 below shows the percent of benzoyl peroxide and micrograms recovered in the epidermis, stratum corneum, and stratum corneum surface combined after 8 hours.

TABLE 1

| | Total | Total | Total |
|---|---|---|---|
| | Application | Recovery | Recovery |
| Composition | (μG) | (μG) | % |
| 5% Benzoyl Peroxide Composition | 800 | 309 | 38.6% |
| 10% benzoyl peroxide formulation | 1502 | 181 | 12.0% |
| 10% 10% benzoyl peroxide gel | 1464 | 168 | 11.4% |
| 8% benzoyl peroxide formulation | 1004 | 92 | 9.1% |
| 6% benzoyl peroxide hydrous gel | 658 | 68 | 10.3% |
| formulation | | | |

The percentage of benzoyl peroxide recovered was consistently greater in skin compartments with the 5% benzoyl peroxide composition in accordance with the present disclosure than with the other formulations. Table 2 below shows the percent of benzoyl peroxide recovered in the epidermis, stratum corneum, and stratum corneum surface combined after 8 hours.

TABLE 2

| | | | Stratum |
|---|---|---|---|
| | | Stratum | Corneum |
| Composition | Epidermis | Corneum | Surface |
| 5% Benzoyl Peroxide Composition | 3.6 | 14.7 | 14.6 |
| 10% benzoyl peroxide formulation | 1.3 | 5.5 | 5.3 |
| 10% 10% benzoyl peroxide gel | 2.1 | 4.8 | 4.3 |
| 8% benzoyl peroxide formulation | 1.3 | 4.0 | 2.3 |
| 6% benzoyl peroxide hydrous gel | 2.2 | 3.4 | 2.0 |
| formulation | | | |

The microgram recovery of benzoyl peroxide recovered was consistently greater in skin compartments with the 5% benzoyl peroxide composition in accordance with the present disclosure than with the other formulations. Table 3 below shows the microgram recovery of benzoyl peroxide in the epidermis, stratum corneum, and stratum corneum surface combined after 8 hours.

TABLE 3

| | | | Stratum |
|---|---|---|---|
| | | Stratum | Corneum |
| Composition | Epidermis | Corneum | Surface |
| 5% Benzoyl Peroxide Composition | 33.9 | 137.6 | 137.2 |
| 10% benzoyl peroxide formulation | 20.1 | 81.8 | 79.2 |
| 10% 10% benzoyl peroxide gel | 32.0 | 71.4 | 64.2 |
| 8% benzoyl peroxide formulation | 15.9 | 47.9 | 28.1 |
| 6% benzoyl peroxide hydrous gel | 19.6 | 30.4 | 18.0 |
| formulation | | | |

### Discussion

In vitro percutaneous absorption of the 5% benzoyl peroxide composition in accordance with the present disclosure in the epidermis, stratum corneum, and stratum corneum surface was greater than that observed with formulations containing larger dispersed particles in the other products. It is believed that as percutaneous absorption is increased, the formulations in accordance with the present disclosure offer a more efficient bactericidal activity as well as prolong kill time.

### EXAMPLE 24

This example illustrates antimicrobial efficacy of topical benzoyl peroxide compositions in accordance with the present disclosure in skin during a three day study.

### Materials and Methods

A split-face, randomized study of 24 patients was conducted. Patients were randomly divided into two groups of 12 subjects each. Each subject underwent mapping of the forehead and cheeks for location of treatment and sampling. Generally predetermined locations above the eyes were identified and used. Each side of the forehead was treated by a technician and a uniform 0.20 ml amount applied: one side received a commercial 5% benzoyl peroxide gel, or a commercial combination 5% BPO-1% Clindamycin gel, while the opposite side received a 5% benzoyl peroxide composition (meeting the U.S. Pharmacopoeia labeling requirements) in accordance with the present disclosure. Subjects refrained from washing the face after 10 p.m. on the evening prior to test start and the morning of the test to establish baseline P. acnes count and for each count thereafter.

Bacteriological sampling was performed using a cyanoacrylate follicular biopsy technique. For the follicular biopsy, a drop of cyanoacrylate glue was applied on a plastic slide to an area of approximately 1×1 cm. The slide was then pressed against the skin until polymerization occurred and gently peeled off. Under a microscope, 20 of the largest follicular plugs were extracted from across the entire cast surface and transferred to the carrier broth. All samples were subsequently plated onto Brucella agar supplemented with yeast extract, dextrose, and cysteine. Plates were incubated anaerobically at 35° C. to 37° C. for 7 days and colony forming units (cfu) of p. acnes counted. For all samples the density of p. acnes was expressed as log₁₀ cfu(s) per square centimeter (cm²).

Baseline sampling had one cyanoacrylate follicular biopsy on the right and left forehead. At 3 and 8 hours after application, bacteriologic sampling was repeated. Male subjects were instructed not to shave their faces during the test period. Subjects were instructed no to wash their faces until after the 24 hours reading the next day and thereafter washed only with water until the study concluded. Subjects returned at 24, 48, and 72 hours for further sampling, and a total of 6 cyanoacrylate follicular biopsies were conducts on each subject during the 3 day study.

### Results

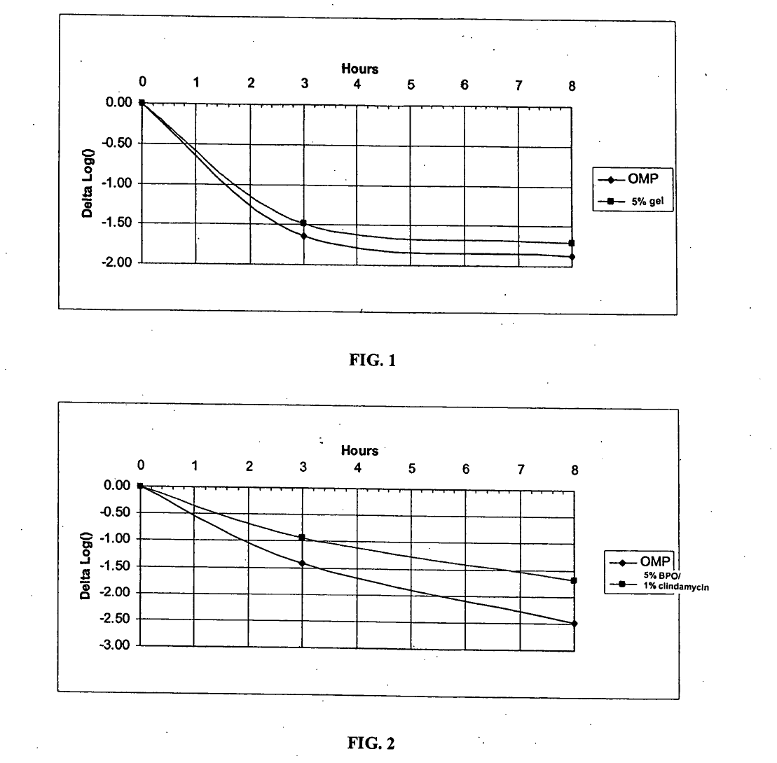

The 5% benzoyl peroxide composition in accordance with the present disclosure yielded a 0.2 log greater reduction in P. acnes than the 5% benzoyl peroxide gel (See for example FIG. 1) and a 0.7 log greater reduction in P. acnes that the 5% benzoyl peroxide/1% clindamycin gel after 3 and 8 hours based on data from follicular biopsies of the forehead. (See for example FIG. 2).

### Conclusion

Benzoyl peroxide compositions in accordance with the present disclosure have been shown to be an effective antimicrobial for acne. A 5% benzoyl peroxide composition in accordance with the present disclosure demonstrated greater in vivo bactericidal activity against P. acnes after a single application during a 72 hour study compared to conventional compositions.

### EXAMPLE25

This example illustrates lesion reduction using topical 5% benzoyl peroxide compositions in accordance with the present disclosure.

### Materials and Methods

Subjects were randomly assigned to receive 5% benzoyl peroxide compositions (meeting the U.S. Pharmacopoeia labeling requirements) in accordance with the present disclosure to the face, or the combination of 5% benzoyl peroxide/1% clindamycin gel. Controlled amounts of study products (0.50 ml) were applied to the face twice daily for 10 weeks. Maps were created to study each subject's forehead and cheeks to ensure precise observations. For all samples, the lesions were expressed as % non-inflammatory lesions and % inflammatory lesions.

### Results

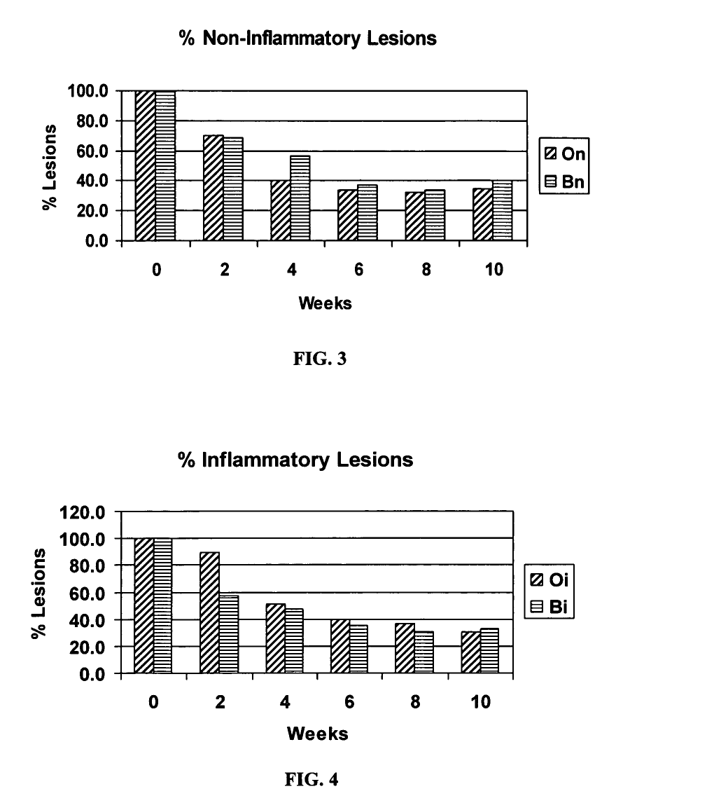

Subject assessment revealed that all products achieved drop in acne lesions (See for example FIG. 3). Compositions in accordance with the present disclosure achieved lesion reduction similar to that of a combination product having both benzoyl peroxide and antibiotic. (See for example FIG. 4).

### Conclusions

Benzoyl peroxide compositions in accordance with the present disclosure have been shown to be an effective therapy for treating acne lesions. The compositions in accordance with the present disclosure demonstrated lesion reduction similar to products containing both antimicrobial and benzoyl peroxide in combination.

It will be understood that various modifications may be made to the embodiments disclosed herein. Therefore, the above description should not be construed as limiting, but merely as exemplifications of embodiments. Those skilled in art will envision other modifications within the scope and spirit of the claims appended hereto.

What is claimed is:

1. A composition comprising a solution of benzoyl peroxide in one or more solvents.

2. A composition as in claim 1 further comprising an additional active ingredient effective in treating acne.

3. A composition as in claim 1 wherein the one or more solvents are selected from the group consisting of:
short chain alkyl esters of benzoic acid, benzyl alcohol, salicylic acid, phenol or phathalic acid;
short chain alkyl ethers of benzoic acid, benzyl alcohol, salicylic acid, phenol or phathalic acid;
short chain alkyl aldehydes of benzoic acid, benzyl alcohol, salicylic acid, phenol or phathalic acid;
short chain alkyl ketones of benzoic acid, benzyl alcohol, salicylic acid, phenol or phathalic acid;
short chain alkyl alcohols of benzoic acid, benzyl alcohol, salicylic acid, phenol or phathalic acid;
aryl esters of benzoic acid, benzyl alcohol, salicylic acid, phenol and phthalic acid;
aryl ethers of benzoic acid, benzyl alcohol, salicylic acid, phenol and phthalic acid;
aryl aldehydes of benzoic acid, benzyl alcohol, salicylic acid, phenol and phthalic acid;
aryl ketones of benzoic acid, benzyl alcohol, salicylic acid, phenol and phthalic acid; and
aryl alcohols of benzoic acid, benzyl alcohol, salicylic acid, phenol and phthalic acid.

4. A composition according to claim 1 wherein the one or more solvents are selected from the group consisting of alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol and alkyl ethers of phenol.

5. A composition according to claim 1 wherein the one or more solvents are selected from the group consisting of benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzaldehyde, benzyl alcohol, ethyl benzyl alcohol, salicylaldehyde, benzyl salicylate, phenyl tolyl ketone, phenyl benzoate, phenyl ether, dibenzyl ether, benzyl benzoate, benzoic acid, 2-phenyl ethyl ester, and combinations thereof.

6. A composition as in claim 1 further comprising additional constituents, such additional constituents comprising thickeners, rheology modifiers, secondary solvent and combinations thereof.

7. A composition as in claim 6 wherein the secondary solvent is selected from the group consisting of ethanol, acetone, dimethyl isosorbide, glycol ethers of C, to C₆ alcohols with no greater than 2 moles of ethylene oxide, and combinations thereof.

8. A composition as in claim 6 wherein the secondary solvent is selected from the group consisting of glycol ethers of phenol with no greater than 2 moles of ethylene oxide, glycol ethers of methanol with no greater than 2 moles of ethylene oxide, glycol ethers of ethanol with no greater than 2 moles of ethylene oxide, glycol ethers of propanol with no greater than 2 moles of ethylene oxide, and combinations thereof.

9. A clear facial toner comprising a composition in accordance with claim 1.

10. A clear facial serum comprising a composition in accordance with claim 1.

11. A stick comprising a composition in accordance with claim 1.

12. A topical composition comprising a composition in accordance with claim 1.

13. A method of treating acne comprising contacting the skin of a user with a topical formulation comprising a composition in accordance with claim 1.

14. A method of treating acne comprising: topically applying an effective amount of the composition of claim 1 to a person in need thereof.

15. A method of treating skin lesions comprising: topically applying an effective amount of the composition of claim 1 to a person in need thereof.

16. A method of improving the percutaneous absorption of a benzoyl peroxide composition topically applied to the skin of a user, the method comprising:
combining benzoyl peroxide and at least one solvent to provide a benzoyl peroxide/solvent solution; and
topically applying the benzoyl peroxide/solvent solution to skin, wherein percutaneous absorption of the benzoyl peroxide is increased compared to application of the undissolved benzoyl peroxide compositions.

17. A method comprising: combining benzoyl peroxide and at least one solvent to provide a benzoyl peroxide/solvent solution; and
formulating a product suitable for topical application to the skin containing the benzoyl peroxide/solvent solution.

18. The method of claim 17 further comprising adding an aqueous phase to the peroxide solution to form an emulsion.

19. The method of claim 17 wherein the aqueous phase comprises at least one component selected from the group consisting of surfactant, humectant, suspending agent, buffer system, and combinations thereof.

20. A method of treating acne comprising applying a product prepared in accordance with the method of claim 17 to the skin of a subject afflicted with acne.

21. A method of increasing the percutaneous absorption of benzoyl peroxide comprising applying a product prepared in accordance with the method of claim 17 to the skin of a subject.
